(19) United States
(12) Patent Application Publication
(10) Pub. No.: US 2018/0206764 A1
(43) Pub. Date: Jul. 26, 2018

(54) COUGH DETECTION APPARATUS AND NON-TRANSITORY COMPUTER READABLE MEDIUM

(71) Applicant: FUJI XEROX CO., LTD., Tokyo (JP)

(72) Inventors: Takanori OZAWA, Kanagawa (JP); Hiroshi HONDA, Kanagawa (JP); Kazuaki MORITO, Kanagawa (JP); Manabu HAYASHI, Kanagawa (JP)

(73) Assignee: FUJI XEROX CO., LTD., Tokyo (JP)

(21) Appl. No.: 15/708,720

(22) Filed: Sep. 19, 2017

(30) Foreign Application Priority Data
Jan. 23, 2017 (JP) 2017-009696

(51) Int. Cl. A61B 5/08; A61B 7/00; A61B 5/00

(52) U.S. Cl. CPC A61B 5/0823; A61B 7/003; A61B 5/7282; A61B 2503/24; A61B 5/7246; A61B 2562/0219; A61B 5/6891

(57) ABSTRACT

A cough detection apparatus includes a receiving unit and a cough detection unit. The receiving unit receives motion information and sound information of a person who sits on a seat. The motion information and the sound information are transmitted from a motion detector and a sound detector. The motion detector and the sound detector are provided for the seat. The cough detection unit detects a cough of the person on the basis of the received motion information and the received sound information.

### CROSS-REFERENCE TO RELATED APPLICATIONS

This application is based on and claims priority under 35 USC 119 from Japanese Patent Application No. 2017-009696 filed Jan. 23, 2017.

### BACKGROUND

### (i) Technical Field

The present invention relates to a cough detection apparatus and a non-transitory computer readable medium.

### (ii) Related Art

A state detection apparatus that enables a cough to be detected has been recently proposed.

### SUMMARY

According to an aspect of the invention, there is provided a cough detection apparatus including a receiving unit and a cough detection unit. The receiving unit receives motion information and sound information of a person who sits on a seat. The motion information and the sound information are transmitted from a motion detector and a sound detector. The motion detector and the sound detector are provided for the seat. The cough detection unit detects a cough of the person on a basis of the received motion information and the received sound information.

### BRIEF DESCRIPTION OF THE DRAWINGS

Exemplary embodiments of the present invention will be described in detail based on the following figures, wherein:

FIG. 1 is a diagram illustrating an exemplary schematic configuration of a cough detection system according to a first exemplary embodiment of the present invention;

FIG. 2 is a block diagram illustrating an exemplary control system of the cough detection system illustrated in FIG. 1;

FIG. 3 is a diagram illustrating exemplary seat configuration data;

FIG. 4 is a diagram illustrating an exemplary detection result table;

FIG. 5 is a diagram illustrating an exemplary probability table;

FIG. 6A is a diagram illustrating an exemplary waveform of acceleration at a seating surface;

FIGS. 6B and 6C are diagrams illustrating exemplary time-series waveforms of sound signals;

FIGS. 7A and 7B are diagrams illustrating exemplary waveforms representing, for each frequency, the amplitudes of sound signals;

FIG. 8 is a diagram illustrating an exemplary display of detection results;

FIG. 9 is a flowchart of exemplary operations of the cough detection system illustrated in FIG. 1;

FIG. 10 is a block diagram illustrating an exemplary control system of a cough detection system according to a modified example of the present invention;

FIG. 11 is a diagram illustrating an exemplary schematic configuration of a cough detection system according to a second exemplary embodiment of the present invention;

FIG. 12 is a block diagram illustrating an exemplary control system of the cough detection system illustrated in FIG. 11;

FIG. 13A is a diagram illustrating an exemplary boarding record table;

FIG. 13B is a diagram illustrating an exemplary detection result table;

FIG. 13C is a diagram illustrating an exemplary probability table;

FIG. 13D is a diagram illustrating an exemplary infectious-disease information table; and

FIG. 14 is a flowchart of exemplary operations of the cough detection system illustrated in FIG. 11.

### DETAILED DESCRIPTION

Exemplary embodiments of the present invention will be described below with reference to the drawings. In the drawings, components having substantially the same functions are designated with identical reference numbers, and repeated description will be avoided.

### Overview of Exemplary Embodiments

A cough detection apparatus according to the exemplary embodiments includes a receiving unit, a cough detection unit, and a specifying unit. The receiving unit receives motion information and sound information of persons who sit on seats, from motion detectors and sound detectors provided for the seats. The motion information and the sound information are transmitted along with identification information of the seats. The cough detection unit detects coughs of the persons on the basis of the received motion information and the received sound information. The specifying unit specifies the seats of persons from whom the coughs are detected.

The seats are disposed in a room, such as an office or a classroom in a school, in which people are present, or in a moving object, such as a bus, a train, or an airplane, that is taken by people. A seat encompasses a chair and a desk.

Each of the motion detectors detects a motion of a person who sits on a corresponding one of the seats, and obtains motion information. Motions of a person include a slouching motion of the human body, a forward-backward motion of the chest, and a forward-backward motion of the neck which are made when the person coughs. The motion information of a person may be, for example, information indicating displacement of the seating surface of a seat which is produced by a person who makes a motion in a state in which the person sits on the seat.

Each of the sound detectors detects a sound from a person who sits on a corresponding one of the seats, and obtains sound information. Sounds include a voice in conversation and a cough sound produced by a cough. As the sound detector, for example, a microphone may be used. A sound signal is exemplary sound information.

The motion detector and the sound detector are provided for a corresponding one of the seats. That is, the motion detector and the sound detector may be disposed on the seat, or may be provided near the seat, such as forward of or upward of the seat.

The cough detection unit detects a cough of a person on the basis of a result of detection of a coughing motion from the motion information and a result of detection of a cough sound from the sound information. The coughing motion indicates various above-described motions of the human body of a person which are made when the person coughs. The cough sound indicates a sound of coughing produced when a person coughs.

It is desirable to learn in advance which motion information corresponds to a coughing motion, by using a learning function such as deep learning. In addition, it is desirable to learn in advance which sound information corresponds to a cough sound, by using a learning function such as deep learning.

### First Exemplary Embodiment

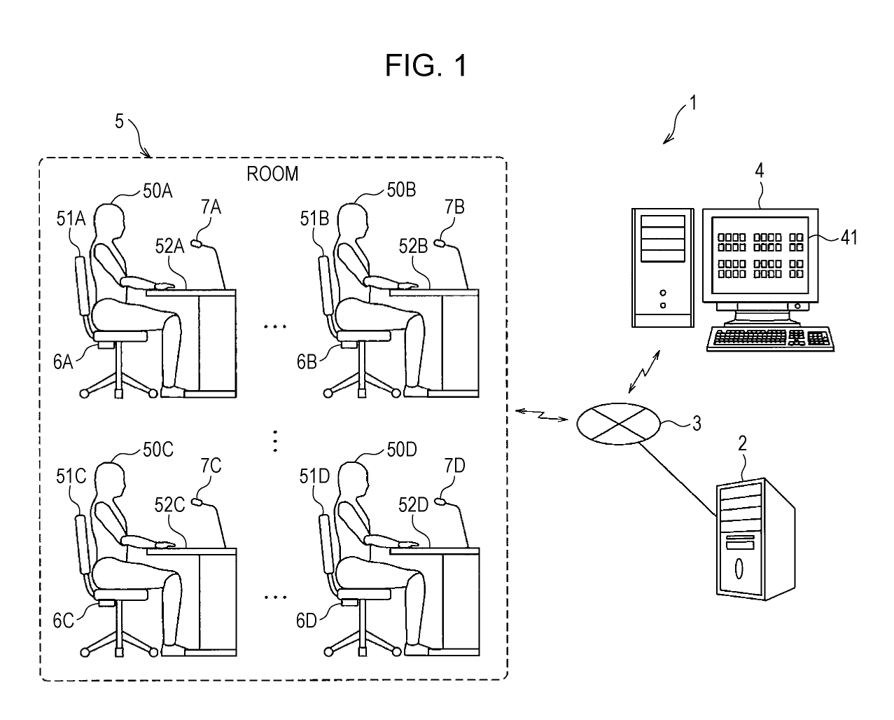

FIG. 1 is a diagram illustrating an exemplary schematic configuration of a cough detection system according to a first exemplary embodiment of the present invention.

A cough detection system 1 identifies the seat of a person who has coughed in a room. A description will be made below by taking an office as the room.

The cough detection system 1 includes a cough detection apparatus 2 that detects a cough. The cough detection apparatus 2 is connected, over a network 3, to motion detectors 6A, 6B, 6C, and 6D, sound detectors 7A, 7B, 7C, and 7D, and an administrator terminal 4 which are disposed in a room 5. The motion detectors 6A, 6B, 6C, and 6D and the sound detectors 7A, 7B, 7C, and 7D are collectively referred to as motion detectors 6 and sound detectors 7, respectively. The administrator terminal 4 may be provided outside the room 5.

In the room 5, chairs 51A, 51B, 51C, and 51D (hereinafter collectively referred to as chairs 51) and desks 52A, 52B, 52C, and 52D (hereinafter collectively referred to as desks 52) which are used by target persons 50A, 50B, 50C, and 50D (hereinafter collectively referred to as target persons 50) such as workers are disposed. A target person 50 is an exemplary person, and a chair 51 is an exemplary seat.

For example, a personal computer, a tablet terminal, or a multifunction portable telephone (smartphone) may be used as the administrator terminal 4.

Each of the motion detectors 6 is provided, for example, for a corresponding one of the chairs 51 used by a target person 50. The motion detector 6 detects displacement of the seating surface which is produced by a motion of the body of the target person 50, for example, by using an acceleration sensor. Specifically, the motion detector 6 detects acceleration at the seating surface in a predetermined direction by using the acceleration sensor, and wirelessly transmits the detection result as an acceleration signal over the network 3 to the cough detection apparatus 2. The acceleration signal is exemplary motion information of a person and an exemplary output signal from the acceleration sensor. The motion detector 6 may be connected to the network 3 in a wired manner.

Each of the sound detectors 7 is provided, for example, on a corresponding one of the desks 52. The sound detector 7 detects a sound emitted from a target person 50, for example, by using a microphone, and wirelessly transmits the detection result as a time-series sound signal over the network 3 to the cough detection apparatus 2. The sound detector 7 may be connected to the network 3 in a wired manner.

As described above, both of the sound detector 7 and the motion detector 6 are provided for the chair 51 and the desk 52 used by a target person 50. Therefore, compared with a case in which the sound detector 7 and the motion detector 6 are directly attached to (worn on) the body of the target person 50, a time and an effort to attach the sound detector 7 and the motion detector 6 to the body of the target person 50 are saved, and inconvenience of the target person 50 having the sound detector 7 and the motion detector 6 attached to their body is avoided.

The network 3 is, for example, a local area network (LAN) or the Internet, and may be wired or wireless.

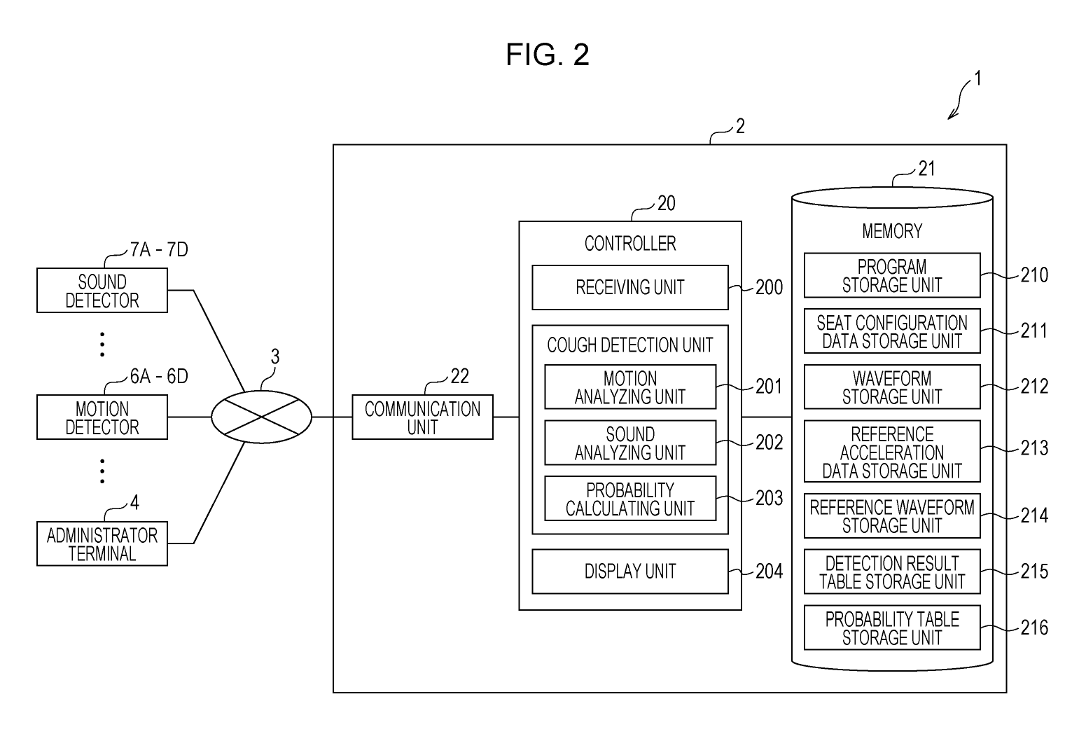

FIG. 2 is a block diagram illustrating an exemplary control system of the cough detection apparatus 2 illustrated in FIG. 1. As illustrated in FIG. 2, the cough detection apparatus 2 includes a controller 20 that controls the units of the cough detection apparatus 2, a memory 21 that is used to store various data, and a communication unit 22 that communicates with the administrator terminal 4, the motion detectors 6, and the sound detectors 7 over the network 3.

The controller 20 of the cough detection apparatus 2 includes a central processing unit (CPU) and an interface. The controller 20 operates in accordance with programs stored in a program storage unit 210 in the memory 21 which is described below. Thus, the controller 20 functions as a receiving unit 200, a motion analyzing unit 201, a sound analyzing unit 202, a probability calculating unit 203, a display unit 204, and the like. The motion analyzing unit 201, the sound analyzing unit 202, and the probability calculating unit 203 are an exemplary cough detection unit. The display unit 204 is an exemplary specifying unit. The units 200 to 204 will be described in detail below.

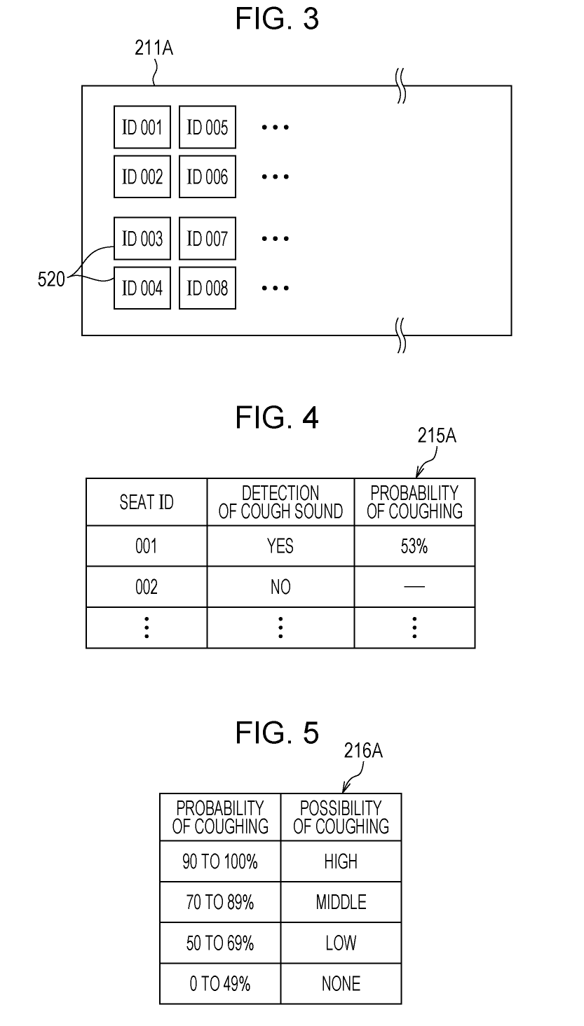

The memory 21 is constituted by a read only memory (ROM), a random access memory (RAM), and the like. The memory 21 includes the program storage unit 210 that stores programs, a seat configuration data storage unit 211 that stores seat configuration data 211A (see FIG. 3), a waveform storage unit 212 that stores sound waveforms, a reference acceleration data storage unit 213 that stores reference acceleration data, a reference waveform storage unit 214 that stores reference waveforms, a detection result table storage unit 215 that stores a detection result table 215A (see FIG. 4), and a probability table storage unit 216 that stores a probability table 216A (see FIG. 5).

In the seat configuration data 211A, a seat diagram according to the layout of the seats (for example, the desks 52) in the room 5 (see FIG. 1), and seat IDs for identifying the seats are stored. A seat ID is exemplary identification information.

The waveforms stored in the waveform storage unit 212 include a waveform representing, for each frequency, the amplitude of a sound signal that is output from a sound detector 7.

The reference acceleration data stored in the reference acceleration data storage unit 213 includes predetermined thresholds (Aₜₕ and −Aₜₕ) for acceleration indicated by an acceleration signal.

The waveforms stored in the reference waveform storage unit 214 include a waveform representing, for each frequency, the amplitude of a sound signal that serves as reference for checking.

The receiving unit 200 receives an acceleration signal transmitted from a motion detector 6 along with a seat ID, and a sound signal transmitted from a sound detector 7 along with a seat ID. In addition, the receiving unit 200 outputs the acceleration signal to the motion analyzing unit 201, and outputs the sound signal to the sound analyzing unit 202.

The motion analyzing unit 201 analyzes an acceleration signal that is output from the receiving unit 200, and determines whether or not a coughing motion is detected. When the motion analyzing unit 201 detects a coughing motion, the motion analyzing unit 201 outputs, to the receiving unit 200, a sound-signal reception command signal indicating a command for receiving a sound signal.

The sound analyzing unit 202 analyzes a sound signal that is output from the receiving unit 200, and determines whether or not a cough sound is detected. The sound analyzing unit 202 registers information about whether or not a cough sound has been detected, in the detection result table 215A. In addition, the sound analyzing unit 202 stores, in the waveform storage unit 212, a waveform that is obtained through frequency analysis on a sound signal and that represents, for each frequency, the amplitude of the sound signal. When the sound analyzing unit 202 detects a cough sound, the sound analyzing unit 202 outputs, to the probability calculating unit 203, a probability calculation command signal indicating a command for calculating a probability.

The probability calculating unit 203 calculates the probability that the person coughs, on the basis of the probability calculation command signal that is output from the sound analyzing unit 202. The probability calculating unit 203 registers the calculated probability of coughing in the detection result table 215A.

The display unit 204 displays the seats of target persons 50 from whom cough sounds have been detected by the sound analyzing unit 202, on a display surface 41 of the administrator terminal 4 so that the seats may be distinguished from the other seats on the seat diagram containing multiple seats.

The communication unit 22 uses Wi-Fi, BlueTooth®, or the like to receive/transmit signals from/to the administrator terminal 4, the motion detectors 6, and the sound detectors 7.

### Configuration of Seat Configuration Data

FIG. 3 is a diagram illustrating exemplary seat configuration data 211A. In the seat configuration data 211A, a seat diagram that contains multiple thumbnails 520 indicating the desks 52 and that corresponds to the seat layout in the room 5 is stored. To each of the thumbnails 520, the seat ID of a corresponding one of the seats is assigned.

### Configuration of Detection Result Table

FIG. 4 is a diagram illustrating an exemplary detection result table 215A. In the detection result table 215A, a “seat ID” field, a “detection of cough sound” field, and a “probability of coughing” field are provided. For each seat ID, information about whether or not a cough sound has been detected from a person who sits on the seat indicated by the seat ID and a probability of coughing are registered.

In the “seat ID” field, an ID for identifying a seat (a chair 51 or a desk 52) is registered. In the “detection of cough sound” field, as information about whether or not the sound analyzing unit 202 has detected a cough sound, “YES” is registered when the sound analyzing unit 202 has detected a cough sound. When the sound analyzing unit 202 has not detected a cough sound, “NO” is registered. In the “probability of coughing” field, a probability (value) of coughing obtained through calculation performed by the probability calculating unit 203 is registered. In the example in FIG. 4, an integer whose unit is percent is registered. However, this is not limiting, and a number having a decimal fraction may be registered.

### Configuration of Probability Table

FIG. 5 is a diagram illustrating an exemplary probability table 216A. In the probability table 216A, a “probability of coughing” field and a “possibility of coughing” field are provided.

In the “probability of coughing” field, probability ranges of “0% to 49%, “50% to 69%”, “70% to 89%”, and “90% to 100%” which are obtained by dividing the probability from 0% to 100% into four sections are registered. In the “possibility of coughing” field, words that indicate information about the magnitudes of possibilities of coughing and that correspond to the probability ranges are registered. For example, “none” is registered for “0% to 49%; “low”, for “50% to 69%”; “middle”, for “70% to 89%”; and “high”, for “90% to 100%”.

### Operations in First Exemplary Embodiment

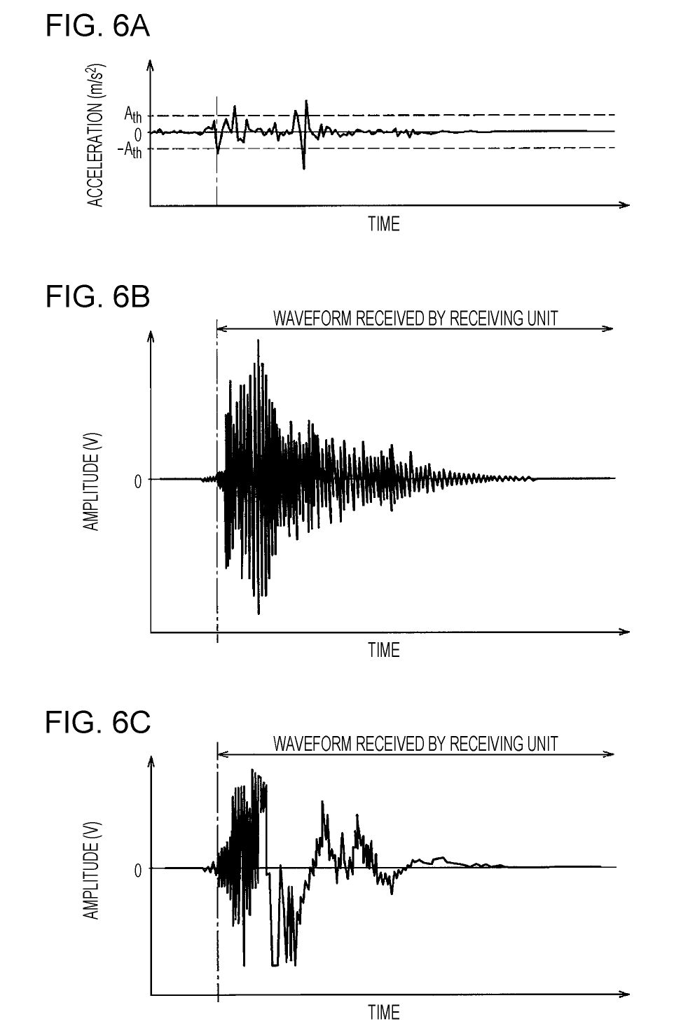

Exemplary operations of the cough detection system 1 will be described with reference to FIGS. 6A to 9.

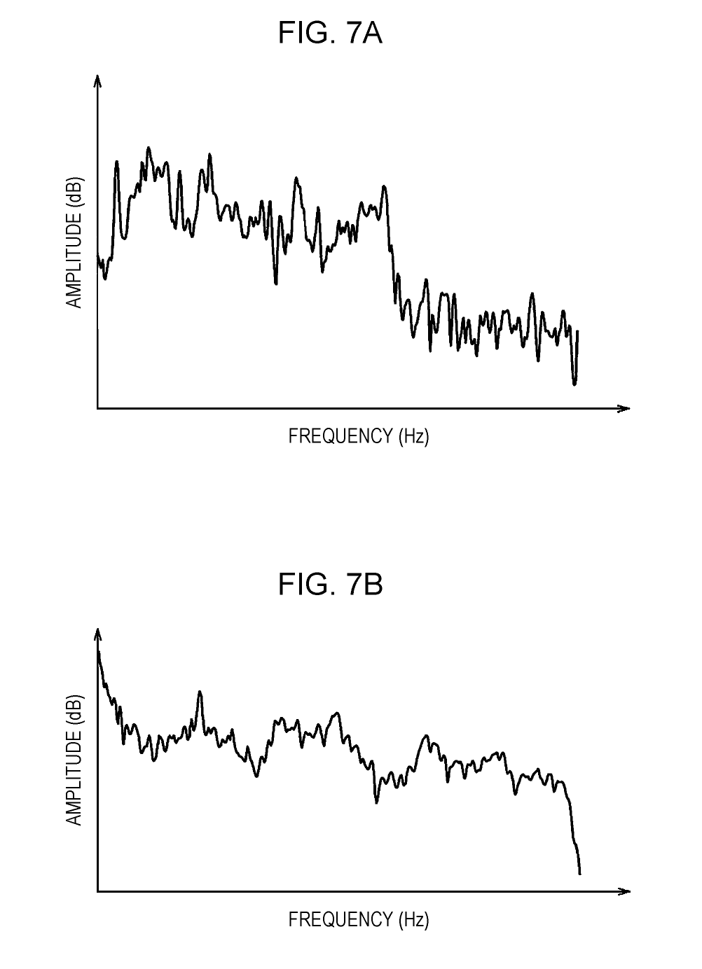

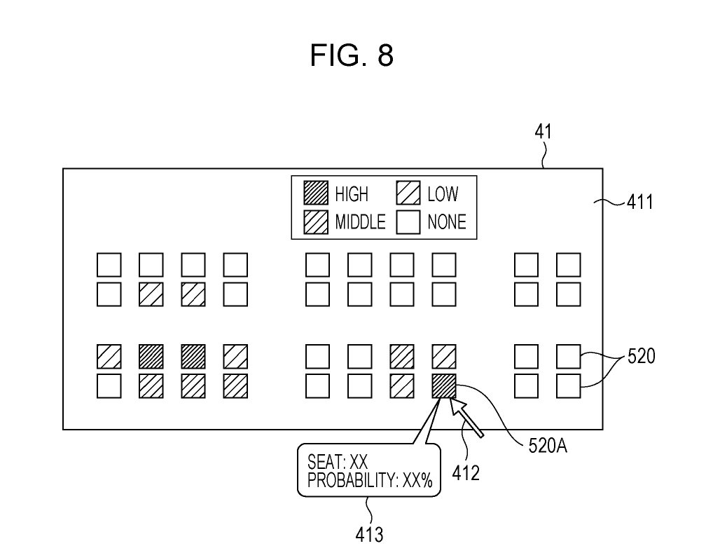

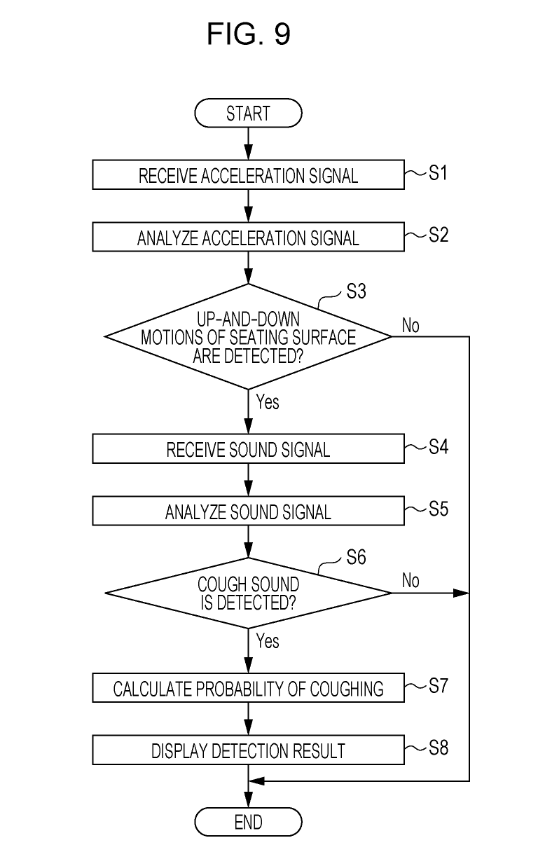

FIG. 6A is a diagram illustrating an exemplary waveform of acceleration at a seating surface. In FIG. 6A, the horizontal axis represents time and the vertical axis represents acceleration at the seating surface of a chair 51 which is produced in the vertical direction. FIG. 6B is a diagram illustrating an exemplary time-series waveform of a sound signal obtained when a person coughs. FIG. 6C is a diagram illustrating an exemplary time-series waveform of a sound signal obtained when a person sneezes. In both of FIGS. 6B and 6C, the horizontal axis represents time and the vertical axis represents the amplitude of a sound signal. FIG. 7A is a diagram illustrating an exemplary waveform representing, for each frequency, the amplitude of a sound signal illustrated in FIG. 6B. FIG. 7B is a diagram illustrating an exemplary waveform representing, for each frequency, the amplitude of a sound signal illustrated in FIG. 6C. In both of FIGS. 7A and 7B, the horizontal axis represents time and the vertical axis represents amplitude (dB). FIG. 8 is a diagram illustrating an exemplary display of detection results. FIG. 9 is a flowchart of exemplary operations of the cough detection system 1.

Each of the motion detectors 6 provided for a corresponding one of the chairs 51 detects displacement of the seating surface of the chair 51, converts the displacement into an acceleration signal, and transmits the acceleration signal along with the seat ID over the network 3 to the cough detection apparatus 2. Each of the sound detectors 7 provided for a corresponding one of the desks 52 detects a sound emitted from a target person 50, converts the sound into a time-series sound signal, and transmits the sound signal along with the seat ID over the network 3 to the cough detection apparatus 2.

The receiving unit 200 receives an acceleration signal transmitted along with the seat ID from a motion detector 6 (S1). The amplitude of the acceleration signal corresponds to the magnitude of acceleration of a motion of the seating surface.

The motion analyzing unit 201 analyzes the acceleration signal (S2). That is, the motion analyzing unit 201 determines whether or not up-and-down motions of the seating surface are detected on the basis of the state in which, as illustrated in FIG. 6A, acceleration in the vertical direction exceeds the predetermined thresholds (Aₜₕ and −Aₜₕ) stored in the reference acceleration data storage unit 213 (S3).

If the motion analyzing unit 201 detects up-and-down motions of the seating surface (Yes in S3), the motion analyzing unit 201 outputs a sound-signal reception command signal to the receiving unit 200.

When the receiving unit 200 receives the sound-signal reception command signal from the motion analyzing unit 201, the receiving unit 200 receives a sound signal transmitted along with the seat ID from the corresponding sound detector 7 for a certain time (for example, ten seconds) (S4). The receiving unit 200 outputs, to the sound analyzing unit 202, a sound analysis command signal indicating a command for analyzing a sound. When a coughing motion is detected, an operation of receiving a sound signal is performed. Thus, it is not necessary to analyze a sound signal every time, enabling unnecessary analysis to be avoided.

The sound analyzing unit 202 analyzes the sound signal received by the receiving unit 200 on the basis of the sound analysis command signal that is output from the receiving unit 200 (S5). That is, the sound analyzing unit 202 performs frequency analysis on the sound signal received by the receiving unit 200, and determines whether or not a cough sound is detected on the basis of the analysis result (S6). Specifically, the sound analyzing unit 202 performs frequency analysis on the sound signal that is output by the sound detector 7, and obtains a waveform representing, for each frequency, the amplitude of the sound signal. Then, the sound analyzing unit 202 checks the obtained waveform against a reference waveform that represents, for each frequency, the amplitude of a sound signal and that is stored in the reference waveform storage unit 214, and obtains the degree of similarity between the waveforms. The sound analyzing unit 202 detects a cough sound when the obtained degree of similarity exceeds a threshold.

For example, in the case of a sound produced by a cough, the sound analyzing unit 202 converts a sound signal received by the receiving unit 200 into a digital signal, and performs frequency analysis such as fast Fourier transform (FFT) on the resulting signal. Thus, a waveform representing, for each frequency, the amplitude of a sound signal as illustrated in FIG. 7A is obtained from a time-series waveform of a sound signal as illustrated in FIG. 6B.

In the case of a sound produced by a sneeze, the sound analyzing unit 202 converts a sound signal received by the receiving unit 200 into a digital signal, and performs frequency analysis such as fast Fourier transform (FFT) on the resulting signal. Thus, a waveform representing, for each frequency, the amplitude of a sound signal as illustrated in FIG. 7B is obtained from a time-series waveform of a sound signal as illustrated in FIG. 6C.

The sound analyzing unit 202 stores the above-described waveform representing, for each frequency, the amplitude of the sound signal in the waveform storage unit 212. The sound analyzing unit 202 registers information about whether or not a cough sound has been detected, in the “detection of cough sound” field in the detection result table 215A which corresponds to the seat ID. In the case of the waveform of a sound signal produced by a cough which is illustrated in FIG. 6B, the degree of similarity between the reference waveform and the waveform that is obtained by performing frequency analysis on the waveform for a cough and that is illustrated in FIG. 7A is large. Therefore, the sound analyzing unit 202 identifies the waveform as a cough sound. In contrast, in the case of the waveform of a sound signal produced by a sneeze which is illustrated in FIG. 6C, the degree of similarity between the reference waveform and the waveform that is illustrated in FIG. 7B and that is obtained by performing frequency analysis on the waveform for a sneeze is small. Therefore, the sound analyzing unit 202 does not identify the waveform as a cough sound.

The motion analyzing unit 201 may identify a coughing motion by checking a reference waveform against the time-series waveform of an acceleration signal received by the receiving unit 200.

When the sound analyzing unit 202 identifies a cough sound, the sound analyzing unit 202 registers “YES” in the “detection of cough sound” field in the detection result table 215A. The sound analyzing unit 202 outputs the seat ID and a probability calculation command signal to the probability calculating unit 203.

When the probability calculating unit 203 receives the seat ID and the probability calculation command signal from the sound analyzing unit 202, the probability calculating unit 203 calculates a probability of coughing (S7). For example, the probability calculating unit 203 calculates, as a probability of coughing of the person, the degree of similarity between the waveform that is stored in the waveform storage unit 212 and that represents, for each frequency, the amplitude of a sound signal and the reference waveform that is stored in the reference waveform storage unit 214 and that represents, for each frequency, the amplitude of a sound signal.

The probability calculating unit 203 registers the calculated probability of coughing in the “probability of coughing” field in the detection result table 215A which corresponds to the seat ID that is output from the sound analyzing unit 202. The probability calculating unit 202 outputs the seat ID to the display unit 204.

The display unit 204 displays a seat diagram containing seats at which coughing is detected (S8). That is, on the basis of the detection result table 215A and the probability table 216A, the display unit 204 displays a seat diagram containing seats at which coughing is detected, on the display surface 41 of the administrator terminal 4 so that the seats at which coughing is detected are distinguished from the other seats.

As illustrated in FIG. 8, a seat diagram 411 containing the thumbnails 520 disposed in accordance with the layout of the seats in the room 5 is displayed on the display surface 41 of the administrator terminal 4 (see FIG. 1). For a specified seat (in the example illustrated in FIG. 8, a desk 52), the display unit 204 displays a thumbnail 520 with a hatch pattern on the basis of the probability of coughing of the person who sits on the seat which is stored in the detection result table 215A, and the possibility of coughing that is associated with a probability range in which the probability of coughing is included and that is stored in the probability table 216A. The hatch pattern is not limited to the pattern illustrated in FIG. 8. For example, color-coding or the like may be used.

An administrator operates the administrator terminal 4, displays a pointer 412 on the display surface 41 of the administrator terminal 4, and moves the pointer 412 to a thumbnail 520A indicating a seat. Then, the display unit 204 displays a balloon 413 in which the probability of coughing of the person who sits on the seat is displayed along with the seat ID.

### Modified Example

In the first exemplary embodiment, the example in which the motion analyzing unit 201 is provided for the controller 20 of the cough detection apparatus 2 is described. In a modified example, an example in which the motion analyzing unit 201 is provided for each of the motion detectors 6 will be described. Points different from those in the first exemplary embodiment will be mainly described below.

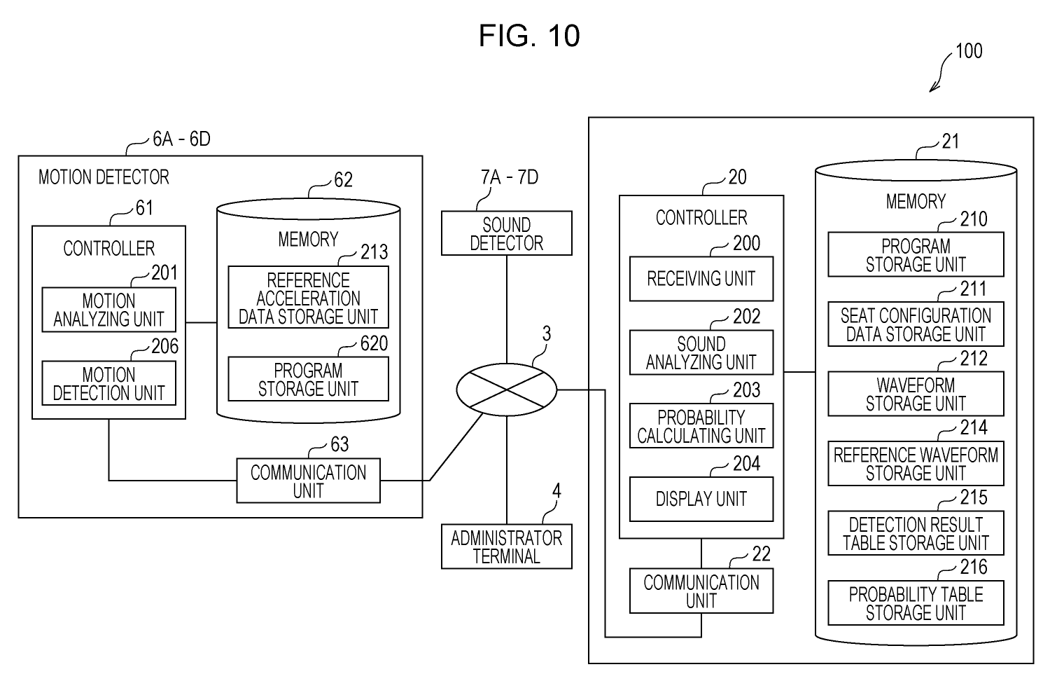

FIG. 10 is a block diagram illustrating an exemplary control system of a cough detection system 100 according to the modified example of the first exemplary embodiment. Each of the motion detectors 6 includes a controller 61, a memory 62, and a communication unit 63. The controller 61 includes a motion detection unit 206 and the motion analyzing unit 201.

The controller 61 of the motion detector 6 includes a CPU and an interface. The controller 61 operates in accordance with programs stored in a program storage unit 620 in the memory 62 so as to function as the motion detection unit 206, the motion analyzing unit 201, and the like.

The memory 62 is constituted by a ROM, a RAM, and the like, and includes the reference acceleration data storage unit 213.

The communication unit 63 uses Wi-Fi, BlueTooth®, and the like to receive/transmit signals from/to the communication unit 22 of the cough detection apparatus 2 described above.

The motion detection unit 206 detects displacement of a seating surface which is produced by a motion of the body of a target person 50, for example, by using an acceleration sensor, converts the displacement into an acceleration signal indicating acceleration at the seating surface in a predetermined direction, and outputs the acceleration signal to the motion analyzing unit 201.

The motion analyzing unit 201 analyzes the acceleration signal, and determines whether or not a coughing motion is detected. When the motion analyzing unit 201 detects a coughing motion, the motion analyzing unit 201 outputs a sound-signal reception command to the receiving unit 200.

When the receiving unit 200 receives the sound-signal reception command from the motion analyzing unit 201 provided for the motion detector 6, the receiving unit 200 receives a sound signal transmitted along with the seat ID from the corresponding sound detector 7, and outputs the sound signal to the sound analyzing unit 202.

### Operations in Modified Example

The motion detection unit 206 obtains the acceleration signal.

The motion analyzing unit 201 determines whether or not acceleration in the vertical direction exceeds the thresholds stored in the reference acceleration data storage unit 213 in the memory 62 of the motion detector 6 so as to detect up-and-down motions of the seating surface.

After that, operations similar to those in steps S4 to S8 illustrated in the first exemplary embodiment are performed. The motion analyzing unit 201 is provided for each of the motion detectors 6, alleviating a burden for the cough detection apparatus 2.

### Second Exemplary Embodiment

In the first exemplary embodiment, the example in which the seat of a person who coughs in a room is identified is described. In a second exemplary embodiment, an example in which the seat of a person who coughs in a moving object is identified will be described. Points different from those in the first exemplary embodiment will be mainly described below.

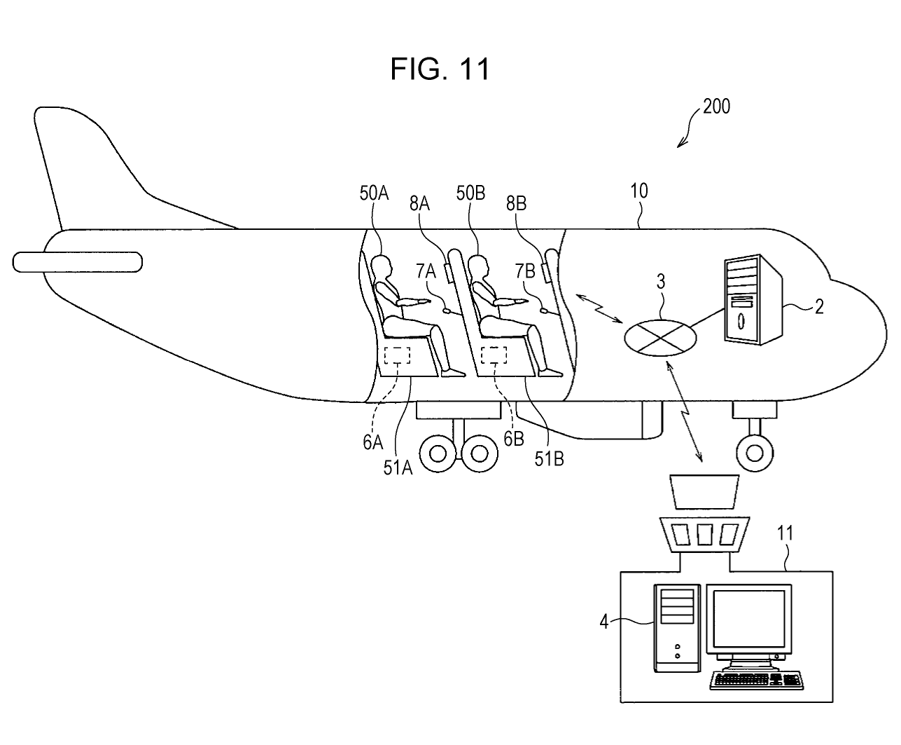

FIG. 11 is a diagram illustrating an exemplary schematic configuration of a cough detection system according to the second exemplary embodiment of the present invention.

A cough detection system 200 identifies the seat of a person who coughs in a moving object 10. A description will be made below by taking an airplane as the moving object 10 and by taking a passenger as a target person 50.

The cough detection system 200 includes pulse detectors 8A and 8B connected to the cough detection apparatus 2 over the network 3, in addition to the cough detection apparatus 2, the network 3, the administrator terminal 4, the motion detectors 6A and 6B, and the sound detectors 7A and 7B. Hereinafter, the pulse detectors 8A and 8B are collectively referred to as pulse detectors 8. The administrator terminal 4 may be provided outside the moving object 10, for example, in a control room 11 such as a control tower.

Each of the pulse detectors 8 provided for a corresponding one of the chairs 51 measures the pulse of a target person 50, for example, by using a camera or an infrared sensor, and wirelessly transmits the pulse information as a pulse signal to the cough detection apparatus 2 over the network 3.

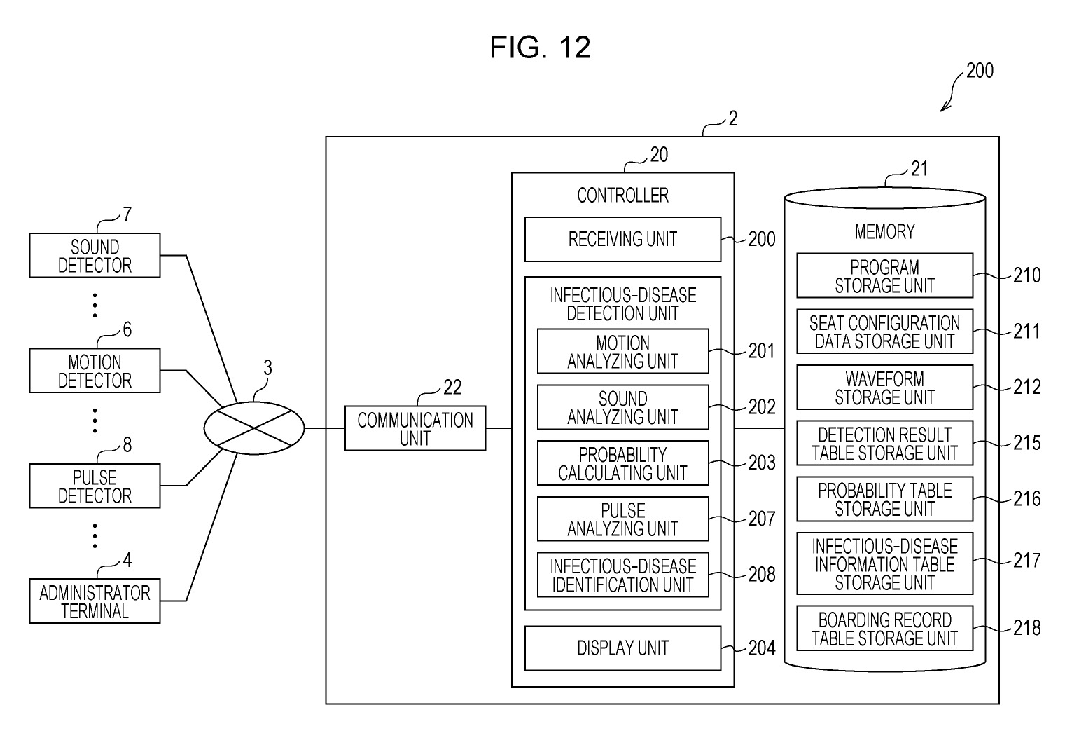

FIG. 12 is a block diagram illustrating an exemplary control system of the cough detection apparatus 2 illustrated in FIG. 11.

The controller 20 operates in accordance with programs stored in the program storage unit 210 so as to function as a pulse analyzing unit 207, an infectious-disease identification unit 208, and the like in addition to the functions described in the first exemplary embodiment. The motion analyzing unit 201, the sound analyzing unit 202, the probability calculating unit 203, the pulse analyzing unit 207, and the infectious-disease identification unit 208 are an exemplary cough detection unit and an exemplary infectious-disease detection unit.

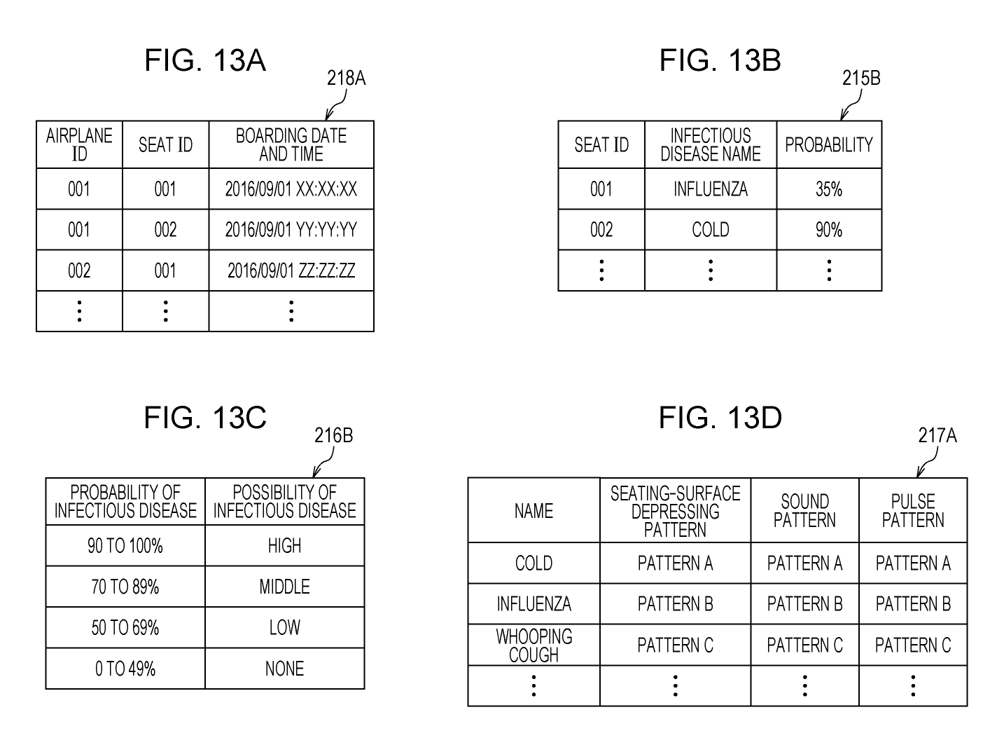

The memory 21 includes an infectious-disease information table storage unit 217 storing an infectious-disease information table 217A (see FIG. 13D) and a boarding record table storage unit 218 storing a boarding record table 218A (see FIG. 13A), in addition to the various storage units described in the first exemplary embodiment.

The receiving unit 200 receives a pulse signal transmitted along with the seat ID from each of the pulse detectors 8, and outputs the pulse signal to the pulse analyzing unit 207.

The motion analyzing unit 201 analyzes an acceleration signal and extracts a seating-surface depressing pattern.

The sound analyzing unit 202 analyzes a sound signal and extracts a sound pattern.

The pulse analyzing unit 207 analyzes a pulse signal and extracts a pulse pattern.

The infectious-disease identification unit 208 detects a cough on the basis of the seating-surface depressing pattern, the sound pattern, and the pulse pattern, and identifies an infectious disease.

### Configuration of Boarding Record Table

FIG. 13A is a diagram illustrating an exemplary boarding record table 218A. In the boarding record table 218A, an “airplane ID” field, a “seat ID” field, and a “boarding date and time” field are provided.

In the “boarding date and time” field, the date and time when a target person 50 gets aboard and sits on the seat indicated by a “seat ID” is registered.

### Configuration of Detection Result Table

FIG. 13B is a diagram illustrating an exemplary detection result table 215B. In the detection result table 215B, an “infectious disease name” field is provided instead of the “detection of cough sound” field in the detection result table 215A described in the first exemplary embodiment. Without removing the “detection of cough sound” field, the “infectious disease name” field may be provided.

### Configuration of Probability Table

FIG. 13C is a diagram illustrating an exemplary probability table 216B. In the probability table 216B, a “probability of infectious disease” field is provided instead of the “probability of coughing” field in the probability table 216A described in the first exemplary embodiment, and a “possibility of infectious disease” field is provided instead of the “possibility of coughing” field.

### Configuration of Infectious-Disease Information Table

FIG. 13D is a diagram illustrating an exemplary infectious-disease information table 217A. In the infectious-disease information table 217A, a “name” field, a “seating-surface depressing pattern” field, a “sound pattern” field, and a “pulse pattern” field are provided.

In the “name” field, an infectious disease name, such as “cold”, “influenza”, or “whooping cough”, is registered. In the “seating-surface depressing pattern” field, a pattern of depressing of a seating surface which corresponds to the infectious disease indicated in the “name” field is registered. In the “sound pattern” field, a sound pattern corresponding to an infectious disease indicated in the “name” field is registered. In the “pulse pattern” field, a pulse pattern corresponding to an infectious disease indicated in the “name” field is registered.

It is desirable to learn in advance which set of a seating-surface depressing pattern, a sound pattern, and a pulse pattern corresponds to which infectious disease, by using a learning function such as deep learning.

### Operations in Second Exemplary Embodiment

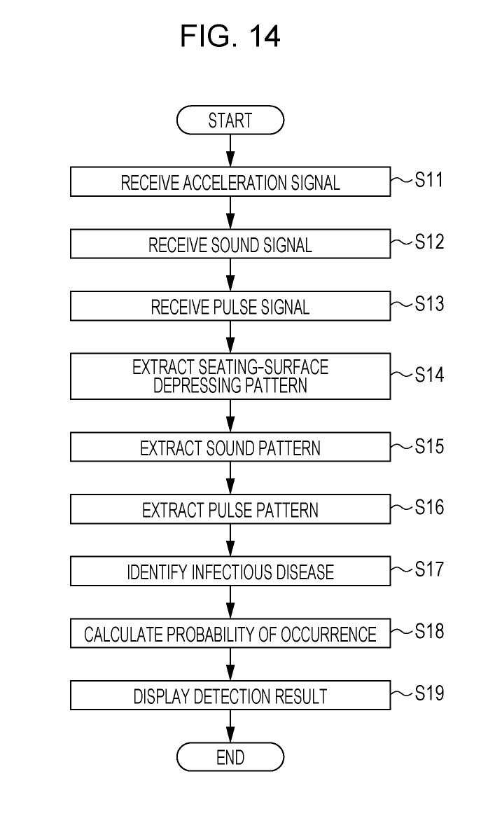

By referring to FIG. 14, exemplary operations of the cough detection system 200 will be described. FIG. 14 is a flowchart of exemplary operations of the cough detection system 200.

The receiving unit 200 receives an acceleration signal from a motion detection unit 6 along with the seat ID (S11), and outputs the seat ID and the acceleration signal to the motion analyzing unit 201. The receiving unit 200 receives a sound signal from the corresponding sound detector 7 along with the seat ID (S12), and outputs the seat ID and the sound signal to the sound analyzing unit 202. The receiving unit 200 receives a pulse signal from the corresponding pulse detector 8 along with the seat ID (S13), and outputs the seat ID and the pulse signal to the pulse analyzing unit 207.

The motion analyzing unit 201 analyzes the acceleration signal received by the receiving unit 200, extracts a seating-surface depressing pattern (S14), and outputs the seating-surface depressing pattern to the infectious-disease identification unit 208.

The sound analyzing unit 202 analyzes the sound signal received by the receiving unit 200, extracts a sound pattern (S15), and outputs the sound pattern to the infectious-disease identification unit 208.

The pulse analyzing unit 203 analyzes the pulse signal received by the receiving unit 200, extracts a pulse pattern (S16), and outputs the pulse pattern to the infectious-disease identification unit 208.

The infectious-disease identification unit 208 detects a cough on the basis of the seating-surface depressing pattern, the sound pattern, and the pulse pattern described above, and identifies an infectious disease. Specifically, the infectious-disease identification unit 208 identifies an infectious disease when all of the degrees of similarity between the seating-surface depressing pattern, the sound pattern, and the pulse pattern described above and the seating-surface depressing pattern, the sound pattern, and the pulse pattern registered in the infectious-disease information table 217A exceed the respective predetermined thresholds (S17). Use of all of the three above-described types of information is not limiting. One or two of these types of information may be used.

The probability calculating unit 203 calculates a probability of occurrence of an infectious disease on the basis of the degrees of similarity described above (S18), and the display unit 204 identifies the seat of the target person 50 who may suffer from the infectious disease, among target persons 50 from whom coughing has been detected, and displays the detection result on the administrator terminal (S19). All of the three types of information, i.e., the seating-surface depressing pattern, the sound pattern, and the pulse pattern, are used in identification of an infectious disease and calculation of a probability of occurrence of the infectious disease. Thus, compared with a case in which one or two types of information are used, an infectious disease may be identified with higher accuracy, and a probability of occurrence may be calculated with high accuracy.

### Modified Example

The exemplary embodiments of the present invention are described above. Exemplary embodiments of the present invention are not limited to the above-described exemplary embodiments. Various changes may be made without departing from the gist of the present invention.

For example, the administrator terminal 4 may include an operation display unit so that the seat configuration data 211A may be rewritten in accordance with a change in the layout of the seats in the room 5. In addition, the probability table 216A may be rewritten.

Some or all of the units of the controller 20 may be implemented by using hardware circuits, such as a field programmable gate array (FPGA) and an application specific integrated circuit (ASIC).

Some of the components in the above-described exemplary embodiments may be removed or changed without departing from the gist of the present invention.

Steps may be, for example, added, deleted, changed, and/or replaced in the flows in the above-described exemplary embodiments without departing from the gist of the present invention. In addition, the programs used in the above-described exemplary embodiments may be provided by recording the programs in a computer-readable recording medium such as a compact disc-read-only memory (CD-ROM). Further, the programs used in the above-described exemplary embodiments may be stored in an external server such as a cloud server, and may be used over a network.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A cough detection apparatus comprising:
a receiving unit that receives motion information and sound information of a person who sits on a seat, the motion information and the sound information being transmitted from a motion detector and a sound detector, the motion detector and the sound detector being provided for the seat; and
a cough detection unit that detects a cough of the person on a basis of the received motion information and the received sound information.

2. The cough detection apparatus according to claim 1,
wherein the cough detection unit detects a cough of the person on a basis of a result of detection of a coughing motion from the motion information and a result of detection of a cough sound from the sound information.

3. The cough detection apparatus according to claim 2,
wherein, after the receiving unit receives the motion information, when the cough detection unit detects a coughing motion from the motion information, the receiving unit starts receiving the sound information.

4. The cough detection apparatus according to claim 2,
wherein the motion detector includes an acceleration sensor that detects, as the motion information, acceleration at a seating surface of the seat.

5. The cough detection apparatus according to claim 3,
wherein the motion detector includes an acceleration sensor that detects, as the motion information, acceleration at a seating surface of the seat.

6. The cough detection apparatus according to claim 4,
wherein the receiving unit receives an output signal from the acceleration sensor, and, when the received output signal exceeds a predetermined threshold, the cough detection unit detects the coughing motion.

7. The cough detection apparatus according to claim 5,
wherein the receiving unit receives an output signal from the acceleration sensor, and, when the received output signal exceeds a predetermined threshold, the cough detection unit detects the coughing motion.

8. The cough detection apparatus according to claim 4,
wherein the receiving unit receives the output signal from the acceleration sensor, and the cough detection unit detects the coughing motion by checking a reference waveform against a time-series waveform of the received output signal.

9. The cough detection apparatus according to claim 5,
wherein the receiving unit receives the output signal from the acceleration sensor, and the cough detection unit detects the coughing motion by checking a reference waveform against a time-series waveform of the received output signal.

10. The cough detection apparatus according to claim 1,
wherein the sound detector outputs a time-series sound signal as the sound information, and
wherein the cough detection unit performs frequency analysis on the time-series sound signal that is output from the sound detector, and detects a cough sound on a basis of the analysis result.

11. The cough detection apparatus according to claim 10,
wherein the cough detection unit detects a cough sound by checking a reference waveform against a waveform obtained through the frequency analysis.

12. The cough detection apparatus according to claim 11,
wherein the cough detection unit calculates a probability corresponding to a degree of similarity between the reference waveform and the waveform obtained through the frequency analysis.

13. The cough detection apparatus according to claim 1, further comprising:
an infectious-disease detection unit that detects an infectious disease of the person on a basis of information from a sensor which detects a living body of the person from whom a cough is detected.

14. A cough detection apparatus comprising:
a receiving unit that receives motion information and sound information of persons who sit on a plurality of seats, the motion information and the sound information being transmitted along with identification information of the plurality of seats from motion detectors and sound detectors, the motion detectors and sound detectors being provide for the plurality of seats;
a cough detection unit that detects coughs of the persons on a basis of the received motion information and the received sound information; and
a specifying unit that specifies seats of persons from whom the coughs are detected.

15. The cough detection apparatus according to claim 14,
wherein the specifying unit displays the specified seats on a seat diagram containing the plurality of seats, the specified seats being displayed in such a manner as to be distinguished from other seats.

16. A non-transitory computer readable medium storing a program causing a computer to execute a process comprising:
receiving motion information and sound information of a person who sits on a seat, the motion information and the sound information being transmitted from a motion detector and a sound detector, the motion detector and the sound detector being provided for the seat; and
detecting a cough of the person on a basis of the received motion information and the received sound information.
